(19) United States
(12) Patent Application Publication
(10) Pub. No.: US 2005/0142216 A1
(43) Pub. Date: Jun. 30, 2005

(54) Method for reversing Alzheimer dementia

(76) Inventor: Keith A. Rindlesbach, Riverton, UT (US)

Correspondence Address: M. Wayne Western;THORPE NORTH & WESTERN, LLP, P.O. Box 1219, Sandy, UT, 84091-1219, US

(21) Appl. No.: 10/750,376

(22) Filed: Dec. 31, 2003

(51) Int. Cl. A61K 31/714; A61K 31/7076; A61K 33/04; A61K 31/60; A61K 31/405; A61K 31/43

(52) U.S. Cl. 424/702; 514/46; 514/52; 514/192; 514/420; 514/165; 514/570

(57) ABSTRACT

A method of reducing the effects of Alzheimer's Dementia can include a step of administering amoxicillin and Vitamin B₁₂ to a patient experiencing Alzheimer Dementia. Another step of the method can include administering indomethacin to the patient. The patient can also receive an administration of S-adenosyl-L-methionine and selinium. It has also been found to be beneficial to include a step of administering ibuprofen and/or aspirin to the patient.

### FIELD OF THE INVENTION

The present invention relates generally to the treatment of diseases. More particularly, the present invention relates to the treatment of Alzheimer's Dementia, a characterization of the symptomatic results of Alzheimer's Disease.

### BACKGROUND OF THE INVENTION

One of the most devastating illnesses afflicting people over the age of 60 is Alzheimer's Disease. This disease does not affect all individuals, but typically affects those in the population that have a genetic susceptibility to it. The incidence of this illness is rising rapidly in society due to a general increase in life expectancy as a result of numerous advances in the medical arts. Because of the overall increase in life expectancy, a greater number of susceptible individuals live to older ages where the disease generally begins to manifest itself. As individuals with the disease age, damage of neural tissue begins in the entorhinal cortex and spreads through the hippocampus and on to the cerebral cortex. Because of the neural locations affected by this devastating illness, individuals experience varying levels of disruption related to memory and reasoning. Affected brains from later stages show immense atrophy of neural tissue and highly enlarged neural ventricles. Due to the nature of this damage, it is, at this time, permanent and irreversible.

Alzheimer's Dementia is a general symptom of Alzheimer's Disease that includes a loss of reason, memory, and judgment. As the disease progresses in afflicted individuals, they become more confused and frustrated by their surroundings, and in many cases become hostile to family members, friends, and caregivers. In many cases, due to damage to the hippocampus, afflicted individuals lose memories acquired more recently in their lives and may begin to remember only those memories acquired in the distant past. They often become difficult to manage, partly due to the confusion and frustration of not being able to remember recent events or the names and faces of those caring for them.

Though the damage done to neural tissue may be irreversible in accordance with present science, success in treating Alzheimer's patients can come from lessening the effects of the associated dementia. It is hoped that by lessening many of the symptoms of this illness that those affected may lead more normal lives, requiring less supervision and expensive medical care.

### SUMMARY OF THE INVENTION

The present invention provides a method of reducing the effects of Alzheimer's Dementia. One step of the method comprises administering amoxicillin and Vitamin B₁₂ to a patient experiencing Alzheimer Dementia. The amoxicillin can be given in the form of Augmentin, a brand of antibiotic that includes two ingredients, namely amoxicillin and clavulanate potassium. Another step of the method can include administering indomethacin to the patient. Yet another step of the method can be to administer S-adenosyl-L-methionine and selenium to the patient. S-adenosyl-L-methionine is often marketed under the name of SAMe. It has also been found to be beneficial to include a step of administering ibuprofen and/or aspirin to the patient.

Additional features and advantages of the invention will be apparent from the detailed description which follows, including specific examples, which together illustrate, by way of example, features of the invention.

### DETAILED DESCRIPTION OF THE PREFERRED EMBODIMENT(S)

Before the present invention is disclosed and described, it is to be understood that this invention is not limited to the particular method steps and materials disclosed herein, because such method steps and materials can vary somewhat. Alterations and further modifications of the inventive steps described herein, and additional applications of the principles of the inventions as described herein, which would occur to one skilled in the relevant art and having possession of this disclosure, are to be considered within the scope of the invention. It is also to be understood that the terminology used herein is used for the purpose of describing particular embodiments only. The terms are not intended to be limiting because the scope of the present invention is intended to be limited only by the appended claims and equivalents thereof.

In describing and claiming the present invention, the following terminology will be used.

The singular forms “a,” “an,” and “the” include plural referents unless the context clearly dictates otherwise. Thus, for example, reference to “a capsule” includes reference to one or more of such materials.

The term “dose” refers to a single administration of a given drug or compound that may be repeated multiple times per day, as warranted by specific embodiments of the present invention. A single dose can include one or multiple units of the administered drug or compound. For example, a 1000 mg dose of a vitamin can be administered in a single 1000 mg capsule or tablet, or in two or more capsules or tablets.

The term “Alzheimer's Disease” refers to a neural degenerative disorder occurring generally throughout the brain, especially in later stages of the disease. Alzheimer's Disease usually starts in the entorhinal cortex and moves to a portion of the limbic system known as the hippocampus. The limbic system is an area of the brain responsible for emotions and instinctive behavior. The hippocampus is involved in memory processing, as more fully described below. From the hippocampus, the Alzheimer's Disease typically moves on to the cerebral cortex. The cerebral cortex processes sensory information, controls voluntary movement, and regulates conscious thought and mental activity. Furthermore, the thalamus and hypothalamus are typically damaged by the disease. The thalamus is responsible for processing emotional and memory related input from the limbic system, and sending it to cerebral cortex. The hypothalamus monitors and corrects internal activity in the body such as food intake, temperature, and the body's internal clock. Alzheimer's Disease as used herein refers to the neural damage occurring in these regions, which is characterized by neural atrophy and highly enlarged neural ventricles. Due to the neural areas involved, the damage done by Alzheimer's disease causes various debilitating behavioral symptoms. These behavioral symptoms increase and are compounded as the disease spreads between neural areas.

The term “Alzheimer's Dementia” is a general characterization of symptoms of Alzheimer's Disease. People experiencing Alzheimer's Dementia often experience a general fear and confusion relating to their surroundings and those taking care of them. This often grows into intense frustration and paranoia. As an example, one neural area associated with the limbic or emotion system of the brain that is severely affected by Alzheimer's Disease is the hippocampus. The hippocampus is responsible for making new memories from recent experiences. When this area of the brain is damaged, the individual affected has difficulty making new memories and, depending on the progression of the disease, they experience memory loss to varying degrees. Often these individuals have trouble distinguishing between reality and their past memories, and often they seem to see their surroundings through their past experiences. This tends to heighten the confusion, because the remembered images of the past do not match with what the individual is experiencing in reality. Also, because of the close interaction between the hippocampus and the limbic system, the hippocampal damage can greatly affect the overall emotion stability of the individual. Furthermore, as the disease progresses from the hippocampus to the cerebral cortex, the person affected begins to lose reasoning skills, the ability to perform simple tasks, and to make sense of sensory input coming from their surrounding environment. This effect is compounded by the previous loss of memory and the overall feelings of frustration and confusion. The disease can also affect the thalamus and hypothalamus, further compounding difficulty in processing of emotion and memory, as well as the regulation of many internal process of the body, such as food intake and temperature control.

The term “about” when referring to a numerical value or range is intended to encompass the values resulting from experimental error that can occur when taking measurements.

Concentrations, amounts, and other numerical data may be expressed or presented herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a dosage range of “0.1 mg to 5 mg” should be interpreted to include not only the explicitly recited dosage of 0.1 mg to 5 mg, but also include individual dosages and the sub-ranges within the indicated range. Thus, included in this numerical range are individual dosages, such as 1 mg, 2 mg, 3 mg, and 4 mg, and sub-ranges, such as from 0.1 mg to 1.5 mg, 1 mg to 3 mg, from 2 mg to 4 mg, from 3 mg to 5 mg, etc. This same principle applies to ranges reciting only one numerical value. For example, a range recited as “less than 5 mg” should be interpreted to include all values and sub-ranges between 0 mg and 5 mg. Furthermore, such an interpretation should apply regardless of the breadth of the range or the characteristics being described.

As used herein, “therapeutically effective amount” refers to at least the minimal amount of a drug, which is sufficient to achieve a desired therapeutic effect. For example, a therapeutically effective amount of “fluconazole” used to treat a fungal infection is at least the minimum amount required in order to reduce the presence of the fungal infection.

It should be noted that the present invention makes no claims as to curing Alzheimer's Disease, only that by following the disclosed therapeutic regimen, the associated cognitive impairment and other symptoms of Alzheimer's Dementia can be lessened. That being stated, one embodiment of the present invention can include a method of reducing the symptoms of Alzheimer's Dementia in a human patient exhibiting the effects of Alzheimer's Disease. In one step of the method, amoxicillin and Vitamin B₁₂ can be administered to the patient. Amoxicillin is an antibiotic used to treat bacterial infections, such as pneumonia, bronchitis, and various infections of mucus membranes. Brand name equivalents of amoxicillin such as Amoxil, Biomox, Polymox, Trimox, Wymox, and other similar antibiotics known to one skilled in the art are deemed to be within the scope of the invention. It can be beneficial to administer the antibiotic clavulanate potassium to the patient, either separately or in combination with amoxicillin. An example of a composition of amoxicillin and clavulanate potassium is marketed under the brand name of Augmentin, which is also considered to be within the scope of the present invention. The amoxicillin and clavulanate potassium can be administered to the patient separately or as a composition, either parenterally or orally. Exemplary forms of administration include a capsule, a tablet, an injection, a swallowable liquid, or a trandermal application. Similarly, Vitamin B₁₂ can be administered to the patient by any of the aforementioned methods.

In one embodiment, a dose of amoxicillin can be administered to the patient in a range of from about 100 mg to about 1750 mg. In another embodiment, an administered dose of amoxicillin can be from about 500 mg to about 1000 mg. These ranges apply to amoxicillin and equivalent name brand antibiotics. Also, an embodiment of the present invention contemplates a dose of clavulanate potassium administered to the patient of from about 50 mg to about 250 mg. An embodiment of the present invention includes a dose range of Vitamin B₁₂ administered to the patient to be from about 100 mg to about 3000 mg. In another embodiment, a dose of Vitamin B₁₂ can be in a range of from about 250 mg to about 2000 mg.

Multiple administrations of amoxicillin and Vitamin B₁₂ can prove beneficial to the patient. It is contemplated to be within the scope of the present invention to give subsequent doses of amoxicillin and Vitamin B₁₂ on at least one other occasion, which is at least 1½ hours after the initial dose. In embodiments including subsequent doses of amoxicillin and Vitamin B₁₂, these doses are given at least 1½ hours apart.

Another step of the method includes administering indomethacin to the patient. Indomethacin is a nonsteroidal anti-inflammatory drug. Brand name nonsteroidal anti-inflammatory drugs such as lndochron E-R, Indocin, and Indocin S-R are included as acceptable indomethacin doses. A typical dose of indomethacin can range from about 25 mg to about 200 mg. Indomethacin can be administered from about 4 hours to about 18 hours after the administration of the first dose of amoxicillin, and can be administered in any form known to one skilled in the art, such as, but not limited to, a capsule, a tablet, an injection, a swallowable liquid, or a transdermal application.

Another step of the present invention includes administering S-adenosyl-L-methionine and selenium to the patient. S-adenosyl-L-methionine is often marketed under the name SAMe. In one embodiment of the present invention, S-adenosyl-L-methionine can be administered from about 4 hours to about 18 hours after the administration of the initial dose of amoxicillin, with a dosage range of from about 50 mg to about 600 mg. In another embodiment, an S-adenosyl-L-methionine dose range can be from about 150 mg to about 300 mg. Selenium can also be co-administered with the S-adenosyl-L-methionine. The amount of selenium administered can be from about 50 mg to about 200 mg. As mentioned above, S-adenosyl-L-methionine and selenium can be administered in any form known to one skilled in the art.

It can also be beneficial to include a step of administering an analgesic/anti-inflammatory, i.e., either ibuprofen or aspirin, to the patient. Ibuprofen can be administered in a range from about 100 mg to about 1000 mg, while aspirin can be administered in a range of from about 125 mg to about 1200 mg. Ibuprofen or aspirin can be administered from about 4 hours to about 18 hours after the administration of the first dose of amoxicillin.

The treatments described in embodiments of the present invention can be enhanced though various means. By preparing or “priming” the body prior to the treatment, the effectiveness of the Alzheimer's Dementia regimen can be greatly increased. Similarly, various additional steps of the method, though not crucial to the successful treatment of the patient, can enhance the effectiveness of the regimen. The following embodiments demonstrate examples of treatment enhancing procedures that can assist in the reversal of Alzheimer's Dementia.

In one embodiment, it can be effective to provide a step of treating the patient for a preexisting fungal infection prior to the commencement of the above mentioned steps of the method. In this embodiment, fluconazole can be administered to the patient in a therapeutically effective amount. Fluconazole is a synthetic triazole antifungal agent, which is often used to treat fungal infections, examples of which are Candida ablicans, Cryptococcal meningitis, and coccidioidomycosis. Fluconazole is designated chemically as 2,4-difluoro-a,a1-bis(1H-1,2,4-triazol-1-ylmethyl) benzyl alcohol. Brand name equivalents of fluconazole include Diflucan, Nizoral, and Sporanox. Any form of administration of flucanozole known to one skilled in the art is considered to be within the scope of the present invention, including tablets, oral suspensions, and injections. Additionally, the administration of a culture of acidophilus to the patient can be beneficial. Acidophilus helps restore the natural bacterial flora in the intestines that may be reduced due to many factors, including the use of antibiotics, improper diet, or fungal infections of the gut. One convenient source of acidophilus is yogurt, however, acidophilus administered in other forms such as capsules or suspension liquids are also considered to be within the scope of the present invention.

It can be beneficial in one embodiment of the invention to administer a cephalosporin antibiotic to the patient. One example of a cephalosporin antibiotic that can prove useful in the treatment is cefdinir, an antibiotic that is marketed under the trade name of Omnicef.

One embodiment of the present invention can include a step of administering nicotinamide adenine dinucleotide (NADH) to the patient. NADH can be administered at any time throughout the treatment procedure, however, it is also preferably given prior to the administration of the first dose of amoxicillin. NADH can be administered in the range of from about 1 mg to about 20 mg. In another embodiment, NADH can be administered in a range of from about 2 mg to about 10 mg. The preferred method of administration of NADH is in the form of a capsule or tablet, however it is conceivable that NADH can be given as an injection, a swallowable liquid, a transdermal application, or any other method known to one skilled in the art.

It can be beneficial in one embodiment of the invention to administer various vitamins and minerals to the patient. Vitamins and minerals that can improve the effectiveness of the Alzheimer's Dementia treatment include, but are not limited to, Vitamins A, B₁, B₂, B₆ and D as well as minerals such as Ca and Mg. Without being bound to any particular theory, it is believed that by providing the patient with a well balanced, low-sugar diet, including a consistent source of at least the USDA recommended daily allowance of vitamins and minerals, that the patient can benefit both from the effects of the vitamins and minerals generally on the body, and from their enhancing effects on the Alzheimer's Dementia reversal treatment.

Yet another embodiment of the present invention can include a step of administering flax oil to the patient. Though this can be accomplished by any means known to one skilled in the art, it is preferable that the flax oil be delivered orally, in the form of a pill or a liquid. The effective dosage of flax oil delivered to the patient can be in a range of about 50 mg to about 3000 mg.

### EXAMPLES

The following examples illustrate embodiments of the invention that are presently known. Thus, these examples should not be considered as limitations of the present invention, but are merely in place to teach the best known methods based upon current experimental data.

### Example 1

### Alzheimer's Dementia Treatment

The following regimen can be used to decrease the debilitating symptoms of Alzheimer's Dementia experienced by a patient as a result of Alzheimer's Disease. After the patient has eaten a low-sugar breakfast, 2000 mg of Vitamin B₁₂ and 875 mg of Augmentin is administered by mouth. Approximately 3 hours after the administration of the Vitamin B₁₂ and Augmentin, the patient is given a 500 mg dose of Vitamin B₁₂ followed by a well-balanced, low-sugar lunch. Approximately 4 hours after ingesting the second Vitamin B₁₂ dose, another 2000 mg of Vitamin B₁₂ is administered along with 500 mg of Augmentin. After another 4 hour period, the patent is given 200 mg of SAMe, 100 mg of selenium, 75 mg of Indocin, and 100 mg of ibuprofen.

### Example 2

### Alternative Alzheimer's Dementia Treatment

Treatment for fungal infections occurs prior to beginning the Alzheimer's Dementia treatment. The patient is treated for possible fungal infections by ingesting 150 mg of Diflucan once per day for 4 days. Following the 4 day period, the patient is fed yogurt containing acidophilus bacteria at least once per day for the remainder of the treatment. Approximately seven days following the end of the fungal treatment, the patient is given 10 mg of NADH to ingest one-half hour before breakfast. Following a low sugar breakfast, the patient ingests 8000 units of Vitamin A and a dietary supplement containing at least the recommended daily allowance of Ca, Mg, and Vitamin D. The patient also ingests a multivitamin supplement containing 15 mg of Vitamin B₁, 17 mg of Vitamin B₂, 200 mg of Niacin, 20 mg of Vitamin B-6, 400 mcg of folic acid, 60 mcg of Vitamin B₁₂, and 100 mg of Pantothenic acid. After waiting 1½ hours, the patient is given 2000 mg of Vitamin B₁₂ and 1000 mg of Augmentin to ingest. After another 3 hours the patient is given 500 mg of Vitamin B₁₂ and 1000 mg of flax oil for ingestion. After another 4 hours the patient is given another 2000 mg of Vitamin B₁₂ and 500 mg of Augmentin, again for ingestion. Following a 4 hour period, the patient is given 200 mg of SAMe, 100 mg of selenium, 75 mg of Indocin, and 100 mg of ibuprofen to ingest. This process can be repeated daily until the effects of Alzheimer's Dementia are noticeably reversed.

It is to be understood that the above-referenced arrangements are illustrative of the application for the principles of the present invention. Numerous modifications and alternative arrangements can be devised without departing from the spirit and scope of the present invention. While the present invention has been described above in connection with the exemplary embodiments of the invention, it will be apparent to those of ordinary skill in the art that numerous modifications can be made without departing from the principles and concepts as set forth in the claims.

1. A method of reducing effects of Alzheimer's Dementia in a patient, comprising steps of:
a) administering a dose of amoxicillin and Vitamin B₁₂ to a patient;
b) administering a dose of indomethacin to the patient;
c) administering a dose of S-adenosyl-L-methionine and selinium to the patient; and
d) administering a dose of at least one of ibuprofen and aspirin to the patient.

2. The method of claim 1, wherein the step of administering S-adenosyl-L-methionine and selenium occurs from about 4 hours to about 18 hours after the administration of amoxicillin.

3. The method of claim 1, wherein the dose of amoxicillin administered to the patient is from about 100 mg to about 1750 mg.

4. The method of claim 3, wherein the dose of amoxicillin administered to the patient is from about 500 mg to about 1000 mg.

5. The method of claim 1, wherein the dose of Vitamin B₁₂ administered to the patient is from about 100 mg to about 3000 mg.

6. The method of claim 5, wherein the dose of Vitamin B₁₂ administered to the patient is from about 250 mg to about 2000 mg.

7. The method of claim 1, wherein the dose of indomethacin administered to the patient is from about 25 mg to about 200 mg.

8. The method of claim 1, wherein the dose of S-adenosyl-L-methionine administered to the patient is from about 50 mg to about 600 mg.

9. The method of claim 8, wherein the dose of S-adenosyl-L-methionine administered to the patient is from about 100 mg to about 400 mg.

10. The method of claim 1, wherein the dose of selenium administered to the patient is from about 50 mg to about 200 mg.

11. The method of claim 1, further comprising a step of administering clavulanate potassium to the patient.

12. The method of claim 1, further comprising a preliminary step of treating the patient for a preexisting fungal infection.

13. The method of claim 12, further comprising step of administering fluconazole to the patient.

14. The method of claim 12, further comprising step of administering a culture of acidophilus to the patient.

15. The method of claim 1, further comprising a preliminary step of administering nicotinamide adenine dinucleotide to the patient prior to administering amoxicillin.

16. The method of claim 1, wherein the step of administering amoxicillin and Vitamin B₁₂ occurs on at least two separate occasions and at least 1½ hours apart.

17. The method of claim 1, further comprising a step of administering a cephalosporin antibiotic to the patient.

18. The method of claim 1, further comprising a step of administering Ca, Mg, and Vitamin D to the patient.

19. The method of claim 1, further comprising a step of administering Vitamin B₁, Vitamin B₂, Vitamin B₆ or combinations thereof to the patient.

20. The method of claim 1, further comprising a step of administering flax oil to the patient in a therapeutically effective amount.
